(19) United States
(12) Patent Application Publication
(10) Pub. No.: US 2015/0192556 A1
(43) Pub. Date: Jul. 9, 2015

(54) CONTINUOUS TURBIDIMETRIC TOTAL IRON MONITORING

(71) Applicant: HACH COMPANY, LOVELAND, CO (US)

(72) Inventors: Michael Sadar, Fort Collins, CO (US); Denton Slovacek, Fort Collins, CO (US)

(21) Appl. No.: 14/417,411

(22) PCT Filed: Jul. 26, 2013
(86) PCT No.: PCT/US13/52332

§ 371 Date: Jan. 26, 2015

Related U.S. Application Data
(60) Provisional application No. 61/676,708, filed on Jul. 27, 2012.

(51) Int. Cl. G01N 33/18

(52) U.S. Cl. CPC G01N 33/1813

(57) ABSTRACT

An embodiment provides a method for determining total iron content in real time in a high purity water system including the steps of: assessing total iron content in each of a plurality of water samples having differing total iron content values; measuring, with a high-intensity turbidimeter, the turbidity values associated with each of the plurality of water samples; identifying a linear relationship between total iron content and turbidity of the plurality of samples; providing a high-intensity turbidimeter positioned in a water conduit of the high purity water system which is responsive to the turbidity of the water system; measuring the turbidity value of the water in real time, with the high-intensity turbidimeter, to generate a continuous data stream representative of the turbidity value; providing a processor programmed with the linear relationship identified between total iron content and turbidity of the plurality of samples; and calculating total iron content of the water in real time by transforming the measured turbidity values of the water using the linear relationship identified between total iron content and turbidity of the plurality of samples by use of the processor. Other embodiments are described.

### CROSS REFERENCE TO RELATED APPLICATIONS

This application claims priority to U.S. Provisional Patent Application No. 61/676,708, entitled “LOW RANGE SUBSTANCE MONITORING”, filed on Jul. 27, 2012, the contents of which are incorporated by reference herein.

### BACKGROUND

The subject matter presented herein generally relates to monitoring of small amounts of substances, as found for example in water.

The constant purity of make-up and condensate water is of utmost importance in high purity water systems such as those found in a high pressure steam generator power plant (also referred to simply as “steam generated power plant”, “power plant”, or “plant”). It is important that the condensate water be free of metals such as iron compounds and any products of corrosion that can cause excessive wear on components, e.g., turbines in a steam generated power plant. It is important to monitor for corrosive materials and iron is a common contaminant which results from corrosion. Failure to remove these metal compounds will lead a breakdown of components, e.g., the turbine surface, and foul membranes, etc. Iron monitoring in steam generated power plants is typically inconsistent and performed on a grab sample basis only.

### BRIEF SUMMARY

In summary, one embodiment provides a method for determining total iron content in real time in a high purity water system comprising the steps of: chemically assessing total iron content in each of a plurality of water samples having differing total iron content values; measuring, with a high-intensity turbidimeter, the turbidity values associated with each of the plurality of water samples; identifying a linear relationship between total iron content and turbidity of the plurality of samples; providing a high-intensity turbidimeter positioned in a water conduit of the high purity water system which is responsive to the turbidity of the water system; measuring the turbidity value of the water in real time, with the high-intensity turbidimeter, to generate a continuous data stream representative of the turbidity value; providing a processor programmed with the linear relationship identified between total iron content and turbidity of the plurality of samples; and calculating total iron content of the water in real time by transforming the measured turbidity values of the water using the linear relationship identified between total iron content and turbidity of the plurality of samples by use of the processor.

In an embodiment, the total iron content in each of a plurality of water samples having differing total iron content values comprise total iron content concentrations of about 50 parts per trillion (ppt)-100 parts per million (ppm).

In an embodiment, the continuous data stream representative of said turbidity value corresponds to turbidity values associated with total iron content concentrations of about 50 parts per trillion (ppt)-100 parts per million (ppm) according to the linear relationship identified.

In an embodiment, the continuous data stream representative of said turbidity value corresponds to turbidity values associated with total iron content concentrations of about 0 parts per billion (ppb)-20 ppb according to the linear relationship identified.

In an embodiment, the continuous data stream representative of said turbidity value corresponds to turbidity values associated with total iron content concentrations of about 0 parts per billion (ppb)-50 ppb according to the linear relationship identified.

In an embodiment, the linear relationship identified takes into account fractional contribution of magnetite and hematite contributions to the total iron content. An embodiment may further include a method of providing an estimate of the fractional contribution of magnetite and hematite to the calculated total iron content of the water.

In an embodiment, providing a high-intensity turbidimeter positioned in a water conduit of said high purity water system which is responsive to said turbidity of the water system may include providing a laser turbidimeter at a water conduit having water exiting a filtration mechanism in a high pressure steam generated power plant. The water conduit having water exiting a filtration mechanism in a high pressure steam generated power plant may include a water conduit leading to a steam formation mechanism in the high pressure steam generated power plant.

In an embodiment, the providing a laser turbidimeter positioned in a water conduit of said high purity water system which is responsive to said turbidity of the water system may include providing the laser turbidimeter at a water conduit having water exiting a condensing mechanism that condenses water from steam.

In an embodiment, a method may include providing a notification responsive to a determination that the calculated total iron content of the water exceeds a predetermined threshold value. A method may further include diverting water of a conduit to an alternative path responsive to a determination that the calculated total iron content of the water exceeds a predetermined threshold value.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example laser turbidimeter.

FIG. 2 illustrates an example of providing a laser turbidimeter for determining low level iron concentrations in a high pressure steam generated power plant.

FIG. 3 illustrates example turbidity measurements correlated with magnetite.

FIG. 4 illustrates an example linear relationship between turbidity measurements and magnetite concentrations.

FIG. 5 illustrates an example electronic device.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail to avoid obfuscation.

High pressure steam generator power plants have a demand for high purity water, necessary to reduce corrosion of the steam conduits up to and leading from the turbines. When corrosion does occur, it results in particulate iron compounds in the form of magnetite (Fe₃O₄) and hematite (Fe₂O₃). If present, these compounds need to be removed through initiation of a reverse osmosis (RO) filtration and ion exchange processes until they no longer can be detected. The current approach for iron detection is to collect a grab sample and perform a digestion to convert the particulate iron to its elemental form. Once in this form, an analysis can be performed using several techniques including ion chromatography or spectrophotometric methods.

There are certain drawbacks to these conventional methods. First, the digestion is lengthy and cumbersome and requires a high degree of skill to perform the analysis. Second, the analysis is not a continuous monitoring process and significant corrosion can take place before it is detected using such periodic measurements. In theory, the water in such power plants always should be exceptionally pure and free of any particulates.

Accordingly, it has been recognized by the inventors that in the context of steam generated power plant water monitoring, conventional techniques are inadequate. Power plants need an online, continuous monitoring of impurities (e.g., the corrosion product in power plant water) but currently none is available. Currently power plants attempting to monitor iron corrosion are limited to the lengthy processes of collecting a sample of the condensate over a period of time then sending the sample to an outside lab for measurement and verification. Again this process is not timely for understanding what is going on in the steam cycle on an “as occurring in-situ” basis.

An example embodiment thus provides for low range monitoring of iron in water. The low range monitoring may be conducted in a real time or near real time fashion such that nearly instantaneous notification of impurities in the water may be had. Such an approach provides greatly improved capabilities for managing water quality in the context of a steam generated power plant, as is needed to protect valuable assets (e.g., turbines).

An example embodiment provides for ultra low range monitoring of iron in steam condensate using high-intensity nephelometry, e.g., as provided by a laser turbidimeter (in this description, laser turbidimeter and laser nephelometer are used interchangeably). Other high-intensity light sources include light-emitting diodes, which are considered an equivalent herein for purposes of this description.

Most iron corrosion in the steam cycle is in particulate form and in very low concentration levels (mg/L concentrations down to several parts per billion (ppb) or even several parts per trillion (ppt)). In an example embodiment, a laser turbidimeter, e.g., a HACH FILTERTRAK660 laser turbidimeter, is installed in-line to a sample of the return condensate and monitors the iron particulates as turbidity. The turbidity value may be correlated against the true iron value of the sample using, e.g., an ultra low range method for determination of total iron. Therefore, the user can now know the iron concentration of power plant condensate water on a real time basis by observing the turbidity value. The user can also monitor the amplitude of fluctuation of the laser turbidity baseline as a threshold to initial presence of iron.

In steam generated power plants steel is often the single or only relevant alloy that contributes any particulates in the condensate. Thus, in such contexts, the particulates in the plant water in question may be considered to be iron from corrosion. Therefore any increase in the nephelometry signal detected by an appropriately placed laser turbidimeter would indicate an increase in iron corrosion. By correlating the nephelometry signal with a direct lab analysis of total iron at the power plant the actual concentration of the iron in the condensate may be determined with the surrogate nephelometry signal. An example turbidimeter that indeed can provide this information is the FILTERTRAK660.

The illustrated example embodiments will be best understood by reference to the figures. The following description is intended only by way of example, and simply illustrates certain example embodiments.

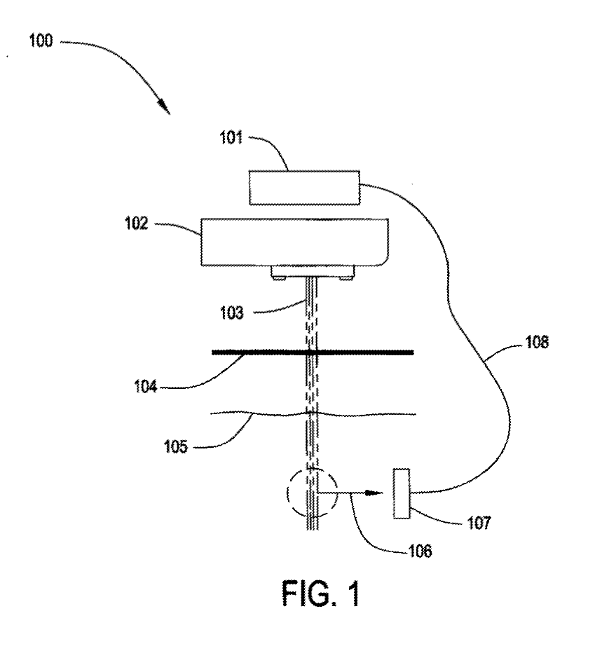

FIG. 1 illustrates an example optical configuration of a laser turbidimeter 100. An example of a turbidimeter 100 that is commercially available is the FILTERTRAK™ 660 laser turbidimeter available from Hach Company of Loveland, Colo. In this embodiment the light source is a 660-nm laser diode module 102 that projects a collimated beam 103 through an aperture 104 into a sample 105, e.g., of condensate water in a power plant. Particulate material that is within the sample 105 will scatter the beam 103 in all directions. A light receiver 107 is positioned, e.g., at a 90-degree angle to the incident light beam 103, to detect a portion of the scattered light 106. Scattered light 106 that reaches the light receiver 107 is then transmitted via an optical fiber 108 to a light detector 101. Alternatively, the scattered light may be collected by an annular optical device described in co-pending U.S. patent application Ser. No. 13/814,669 (U.S. Patent Application Publication 2013/0135613 A1), incorporated by reference in its entirety. The signal may be scaled, e.g., by a processor at or in communication with the light detector 101, to a common calibration standard for turbidity, as described further herein. The laser turbidimeter 100 may be selected for use in the contexts discussed herein (i.e., steam generated power plant condensate water monitoring) because of the high stability and energy density of the incident light source 102, which provides a superior limit of detection when compared to other conventional turbidimeters.

In particular, the laser turbidimeter 100 settings are adjusted to provide the greatest sensitivity to changes in the sample 105 given the low ranges (e.g., ppb) that are to be detected. The flow or presentation of sample 105 to the laser turbidimeter 100 may be set to the low end of a flow range (e.g., 100 ml/minute) to enhance the removal of any bubbles that may form in the sample 105. Mathematical algorithms that are designed to eliminate bubble noise also may be initiated. The signal averaging of the laser turbidimeter 100 may be set to a maximum value to minimize interference from environmental sources such as pump vibrations or the like. The laser turbidimeter 100 may perform a measurement at the rate of once per second, and the value projected to the local display for the laser turbidimeter 100 (not illustrated). The laser turbidimeter 100 may log a measurement value at an appropriate rate, e.g., every minute. The laser turbidimeter 100 may be allowed to run for extended periods, e.g., several weeks, under such conditions.

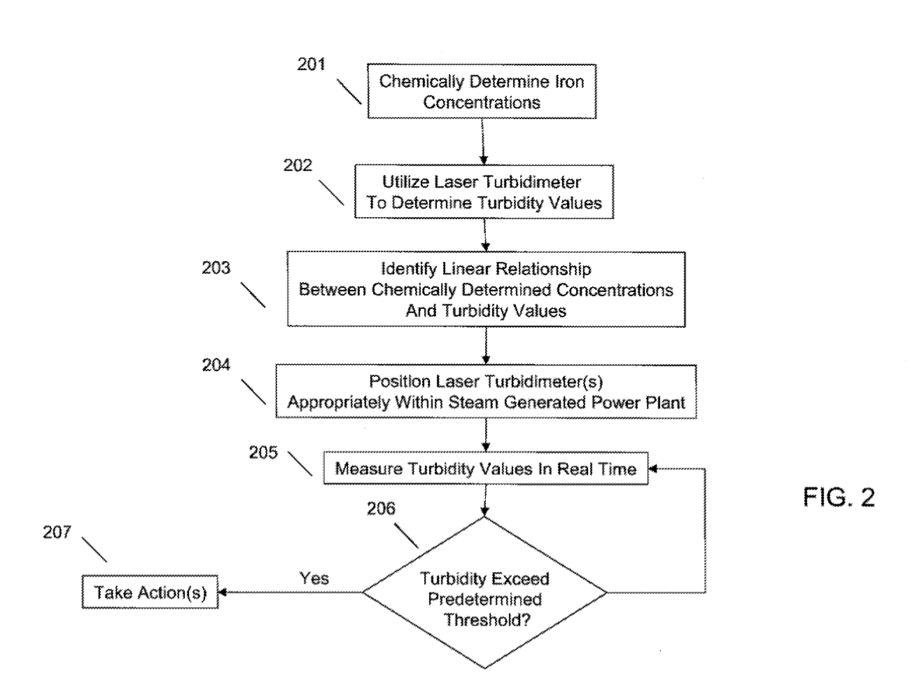

Referring to FIG. 2, an embodiment provides a method for determining total iron content in real time in a high purity water system such as in a high pressure steam generated power plant. As illustrated, a relationship is established between known iron concentrations and the turbidity value registered by a laser turbidimeter. For example, at 201 total iron concentrations may be determined chemically for a particular water conduit in a steam generated power plant. A laser turbidimeter may be used to determine the turbidity value that results for given iron concentrations at 202 (e.g., as chemically determined in 201).

This permits the establishment of a relationship at 203 between the iron concentration and the turbidity values produced by the laser turbidimeter. It has been established, as further described in the examples described herein, that at low concentrations (e.g., up to about 800 ppb), the relationship is linear, whereas at higher concentrations of iron (e.g., exceeding about 7 ppm) the linearity breaks down. It should be noted that for the specific example of the FT660 laser nephelometer, the maximum range in current instrumentation is to about 5000 mNTU. For this type of technology, the response will be linear over the entire range and this correlates to about 800 ug/L total iron. In laser nephelometry, linearity is lost at about 40 NTU (40,000 mNTU) which is a function of the analysis path length for this type of instrument (and other factors such as particle absorption). Thus, if the linear correlation is extended to this higher level, linearity would extend to about 7 ppm, based on the functions described herein. Thus, for low level measurements, e.g., at ppb, a linear relationship may be identified and utilized, as further described herein.

One or more laser turbidimeters are appropriately positioned to measure the water of the power plant in real time at 204. For example, a laser turbidimeter may be positioned to have access to a flow of sample water from a water conduit having water exiting from a filtration or ion exchange mechanism in the plant to monitor iron levels (via turbidity, as further described herein) in order to confirm filtration is adequate, e.g., upstream of an expensive component such as a plant turbine. As another example, a laser turbidimeter may be positioned to have access to a flow of sample water from a water conduit having water that has exited a steam formation mechanism, e.g., after the water has condensed from steam, thus allowing the water conduit's flow to be monitored at a different position. Thus, more than one laser turbidimeter may be appropriately positioned in the power plant to measure iron in conduits of interest.

The laser turbidimeter(s) measure at 205 the water of the conduit(s) in real time, thus providing turbidity values in an on-line or real time fashion. The turbidity values thus provided may be compared to one or more predetermined thresholds at 206, e.g., to determine if the turbidity of the water exceeds the value(s). If so, appropriate action(s) 207 may be taken, such as sounding an alarm, notifying a user that a limit has been exceeded, diverting water within the plant, etc.

By virtue of the linear relationship established at 203, an embodiment may thus use turbidity values measured and compared with predetermined value(s) to determine an iron concentration of the water and take appropriate actions, if necessary. Non-limiting representative examples of determining a linear relationship between laser turbidity values and iron concentrations are given below.

### Representative Examples

Total Iron Assay. The conventional Hach colorimetric dissolved iron procedure (FerroZine® Rapid Liquid Method, Hach Method 8147, Hach Water Analysis Handbook, 5^{th} Ed. (2008)) was modified to provide a new ultra low range (ULR) procedure capable of quantifying 1 ppb total iron with a 30-minute digestion. Suspended and dissolved iron was digested and reduced to the ferrous state using Hach FerroZine® reagent and the Hach® DRB200 digestion block. Ferrous iron complexes with the same FerroZine reagent to produce a purple-colored complex which may be analyzed colorimetrically. The intensity of the FerroZine-iron complex is directly proportional to the total iron concentration of the sample. The low detection limit is achieved through the use of a stable PourThru/Sipper Cell apparatus with a Hach DR6000 or DR3900 spectrophotometer. The Hach ULR Total Iron Analysis assay (LIT2011) is published on the Hach website at http://www.hach.com/filtertrak-660-sc-laser-nephelometer-sensor/product-downloads?id=7640461566&callback=qs, and is incorporated herein by reference in its entirety.

Stock solutions of 250 mg/L (of either magnetite or hematite) were prepared and utilized for testing. These stock solutions were then diluted 1:3000 by volume to yield a concentration that would be spiked, e.g., via peristaltic pump into a clean Reverse Osmosis (“RO”) sample stream. After the RO sample was injected with the particulate iron, it travelled through a mixing loop and then to a laser turbidimeter, e.g., laser turbidimeter 100. The speed on the pump may be held at different rates to simulate different particulate iron concentrations. Thus, several levels of particulate iron, ranging from 1-25 ppb were run through the laser turbidimeter to derive a laser turbidity correlation to the total iron concentration. For each solution of particulate iron, spikes were run from the lowest concentration to the highest concentration to minimize contamination from sample carryover.

Three different stock solutions were run at different concentrations of particulate iron until adequate levels of each were established so a least squares linear correlation could be drawn. This was performed for each form of particulate iron (hematite and magnetite) and then one correlation was developed for a 50:50 mix by mass of the two forms of particulate iron. The laser turbidimeter used in this example was a HACH FILTERTRAK 660 laser turbidimeter, which is outlined in FIG. 1, and employed a 7.5 mW, 670-nm laser diode as a light source. The laser turbidimeter was set up with a 100 ml/minute flow rate to minimize bubble interference and also had both signal averaging and bubble removal algorithms turned on as recommended in the manual. Data was logged at a 6 second interval for the duration.

Each level of particulate iron was run for approximately 60 minutes. This was to allow for enough time for the spike to stabilize in the laser turbidimeter and once this was achieved a minimum of 50 measurements were logged. Once logged, the average, standard deviation and relative standard deviation of the laser turbidimeter value was determined for each spike. Once all the different spikes for a given stock solution of particulate iron were run, a linear-based least squares analysis was derived between the averaged laser turbidity value and the theoretical total iron concentration in the respective spike.

A grab sample of each spiked iron level was taken and a separate total iron analysis was performed according to the previously-described ULR assay. For this, the sample was first digested and then a spectrophotometric analysis was performed as an independent confirmation of the total iron concentration. Throughout the study, this laboratory assay closely correlated to the theoretical values of total iron in the spikes for all samples.

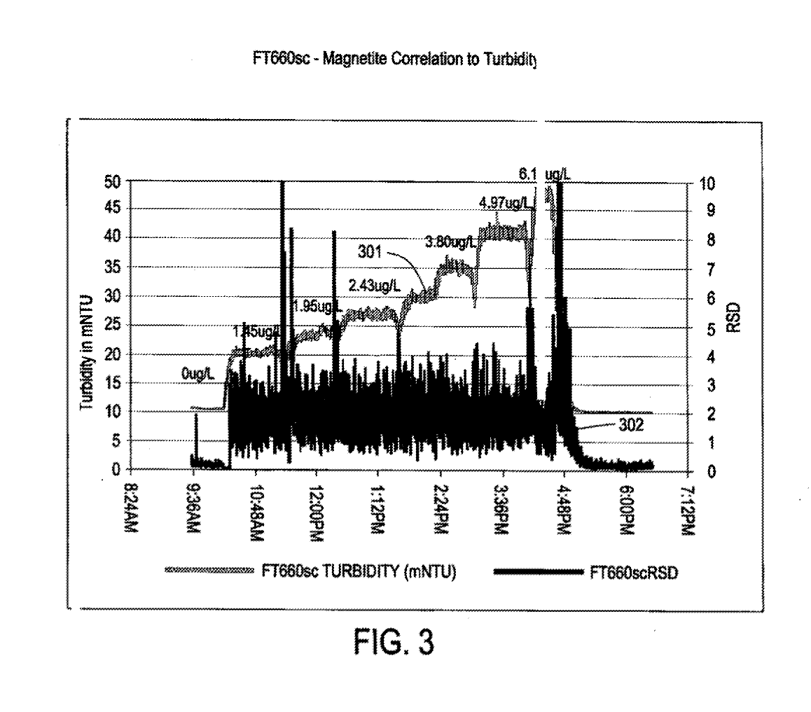

FIG. 3 shows a typical response curve for the laser turbidimeter turbidity value (mNTU) through the different levels of particulate iron. The upper trace 301 represents the turbidity measurement at several different levels of particulate iron (as indicated, 0 ug, 1.45 ug, 1.95 ug, 2.43 ug, 3.80 ug, 4.97 ug, and 6.14 ug). The numerical values above the trace 301 were for the different levels of particulate iron that were generated. The lower trace 302 is the RSD parameter for the instrument, which is a measurement of degree of variability of the measurement baseline. A low RSD value, e.g., below 0.5 RSD, is indicative of a particle free baseline and anything above such a value is indicative of the presence of particles, but at very low concentrations. The RSD value obtained in FIG. 3 is a conformational response to the elevated turbidity values that resulted from the particulate iron.

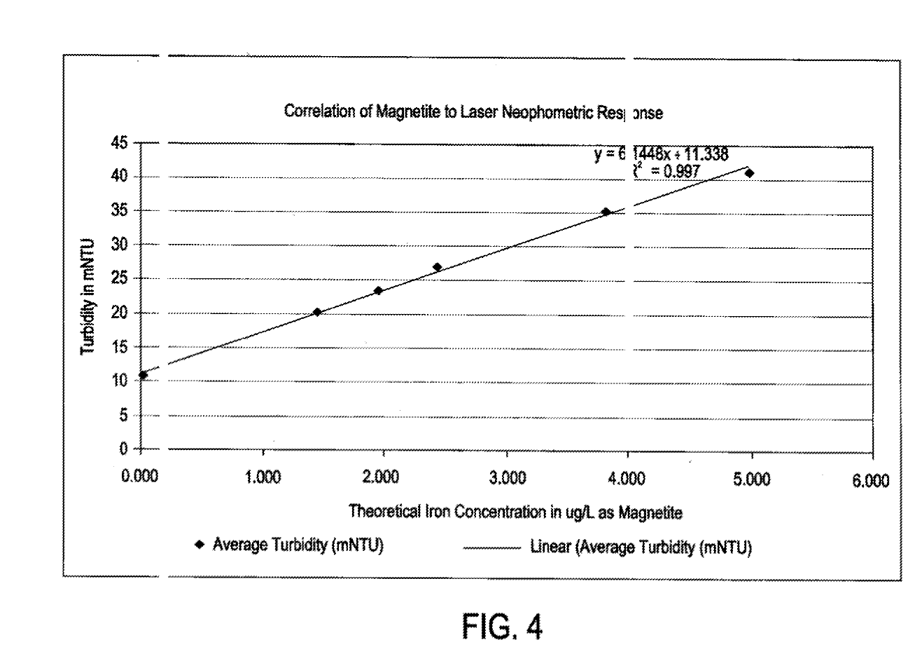

FIG. 4 illustrates the linear correlation or relationship between magnetite iron, expressed as total iron, and laser turbidity measurements. FIG. 4 illustrates that the correlation is highly linear with a correlation coefficient of 0.997. Essentially an increase of 1 ppb in particulate iron resulted in a 6.14 mNTU increase in turbidity. The limit of detection for the HACH FILTERTRAK 660 laser turbidimeter is specified at 0.3 mNTU, which is well below the measurement range for the study of this example. Extrapolation to this limit of detection value yields a detection limit of 48 parts per trillion (ppt). Thus, this laser turbidimeter was capable of detecting this form of particulate iron at the levels described herein.

Similar correlations were also derived for hematite and for the 50:50 mass fraction of hematite-magnetite. The responses to hematite were three times greater than magnetite (again, as illustrated in FIG. 4 as a representative example) and the 50:50 mass fraction spikes had a response that was in between the two separate correlation curves. The explanation for this difference is due to the relative absorbance characteristics of the different compounds of iron. Magnetite, a dark grey material will significantly absorb more of the incident light from the turbidimeter, resulting in a reduced response. Hematite, an orange compound has lower absorbance characteristics and allows for more efficient light scatter from its surface, providing a higher turbidimetric response.

The use of laser turbidimeter(s) to serve as a rapid response and comparative surrogate for particulate iron contamination in steam condensate waters is possible according to the various embodiments described. The correlation between laser nephelometry turbidity values to particulate iron was established for both hematite and magnetite, both common indicators of corrosion in steam generated power plants. However, the response curves for the example studies provided herein are different with respect to their slopes, with the hematite response approximately three times greater than that of the magnetite response curve. In an example steam generated power plant context, the upper limit for particulate iron may be about 10 ppb, which was easily detectable in either form by the laser nephelometry techniques illustrated herein. If applied from a conservative approach, the magnetite parameter may be used as a surrogate measurement for particulate iron and a value that results in a change of 60 mNTU will correspond to 10 ppb iron, which has been demonstrated to be within the measurement capabilities of the HACH FILTERTRAK 660 laser turbidimeter.

As may be appreciated from the examples given above, laser turbidity measurements may be related to iron concentrations at very low levels (e.g., at levels below ppb concentrations). Such laser turbidity measurements may therefore be utilized in real time monitoring of water quality in sensitive contexts, e.g., high pressure steam generated power plants.

It will be understood by those having ordinary skill in the art that various embodiments may be implemented as a system, method, apparatus or program product. Accordingly, various embodiments may take the form of an entirely hardware embodiment, an entirely software embodiment (including firmware, resident software, micro-code, etc.) or an embodiment combining software and hardware aspects. Furthermore, embodiments may take the form of a program product embodied in one or more readable medium(s) having device readable program code embodied therewith.

Any combination of one or more device readable medium(s) may be utilized. The device readable medium may be storage medium. A storage medium may be, for example, but not limited to, an electronic, magnetic, optical, electromagnetic, infrared, or semiconductor system, apparatus, or device, or any suitable combination of the foregoing. A storage medium may be any tangible, non-signal medium that can contain or store a program for use by or in connection with an instruction execution system, apparatus, or device. Information may be communicated (transmitted or received) by an electronic device in accordance with embodiments in a signal medium, which may include a propagated data signal with program code or other information embodied therewith.

Program code for carrying out operations of various embodiments may be written in any combination of one or more programming languages (including an object oriented programming language such as Java™, Smalltalk, C++ or the like and conventional procedural programming languages, such as the “C” programming language or similar programming languages). The program code may execute entirely on a single device, partly on a single device, as a stand-alone software package, partly on a single device and partly on a remote device, or entirely on a remote device. The devices may be connected through any type of network, including a local area network (LAN) or a wide area network (WAN) or the connection may be made to an external computer over the Internet, wired or wirelessly.

It will be understood that certain embodiments can be implemented by a device executing a program of instructions. These program instructions may be provided to a processor of a special purpose device or other programmable data processing apparatus to produce a machine, such that the instructions, which execute via the processor of the device or other programmable data processing apparatus, implement the functions/acts specified.

These program instructions may also be stored in a device readable storage medium that can direct a device or other programmable data processing apparatus to function in a particular manner, such that the instructions stored in the readable storage medium produce an article of manufacture including instructions which implement the function/act specified.

The program instructions may also be loaded onto a device or other programmable data processing apparatus or the like to produce a device implemented process such that the instructions which execute on the device or other programmable apparatus provide processes for implementing the functions/acts specified.

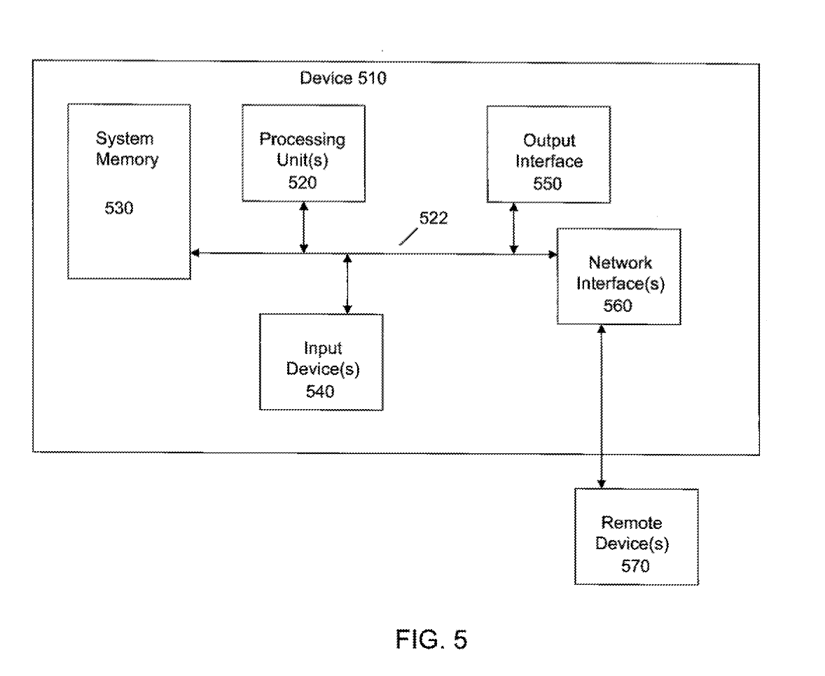

Referring now to FIG. 5, an example device that may be used in connection with one or more embodiments includes a device 510. The device 510 may comprise a laser turbidimeter or a component thereof. Components of device 510 may include, but are not limited to, a processing unit 520, a system memory 530, and a system bus 522 that couples various system components including the system memory 530 to the processing unit 520. Device 510 may include or have access to a variety of readable media. The system memory 530 may include readable storage media, for example in the form of volatile and/or nonvolatile memory such as read only memory (ROM) and/or random access memory (RAM). By way of example, and not limitation, system memory 530 may also include an operating system, application programs, other program modules, and program data.

A user can interface with (for example, enter commands and information) the device 510 through input devices 540. A monitor or other type of device can also be connected to the system bus 522 via an interface, such as an output interface 550. In addition to a monitor, device may also include other peripheral output devices. The device 510 may operate in a networked or distributed environment using logical connections to one or more other remote device(s) 570. The logical connections may include network interface(s) 560 to a network, such as a local area network (LAN), a wide area network (WAN), and/or a global computer network, but may also include other networks/buses.

The device 510 may form part of a laser turbidimeter configured to implement one or more of the steps in the representative methods for using laser technology to monitor low ranges of substances in water, for example iron. Thus, the processing unit(s) 520 may be a processor of a laser turbidimeter, for example included in light detector 101 in FIG. 1. Moreover, the remote device(s) 570 may include a light receiver, e.g., light receiver 107 of FIG. 1 and/or other devices, e.g., a remotely located computing device.

This disclosure has been presented for purposes of illustration and description but is not intended to be exhaustive or limiting. Many modifications and variations will be apparent to those of ordinary skill in the art. The embodiments were chosen and described in order to explain principles and practical application, and to enable others of ordinary skill in the art to understand the disclosure for various embodiments with various modifications as are suited to the particular use contemplated.

Although illustrative embodiments have been described herein, it is to be understood that the embodiments are not limited to those precise embodiments, and that various other changes and modifications may be affected therein by one skilled in the art without departing from the scope or spirit of the disclosure.

What is claimed is:

1. A method for determining total iron content in real time in a high purity water system comprising the steps of:
chemically assessing total iron content in each of a plurality of water samples having differing total iron content values;
measuring, with a high-intensity turbidimeter, the turbidity values associated with each of the plurality of water samples;
identifying a linear relationship between total iron content and turbidity of the plurality of samples;
providing a high-intensity turbidimeter positioned in a water conduit of said high purity water system which is responsive to said turbidity of the water system;
measuring the turbidity value of water in the water conduit in real time, with the high-intensity turbidimeter, to generate a continuous data stream representative of said turbidity value;
providing a processor programmed with the linear relationship identified between total iron content and turbidity of the plurality of samples; and
calculating total iron content of the water in the water conduit in real time by transforming the measured turbidity values of the water using the linear relationship identified between total iron content and turbidity of the plurality of samples by use of the processor.

2. The method of claim 1 wherein said high-intensity turbidimeter comprises a laser for its source of light.

3. The method of claim 1 wherein said high-intensity turbidimeter comprises a light-emitting diode for its source of light.

4. The method of claim 1, wherein said total iron content in each of a plurality of water samples having differing total iron content values comprises total iron content concentrations of from about 50 parts per trillion (ppt) to about 100 parts per million (ppm).

5. The method of claim 4, wherein said continuous data stream representative of said turbidity value corresponds to turbidity values associated with total iron content concentrations of from about 50 parts per trillion (ppt) to about 100 parts per million (ppm) according to the linear relationship identified.

6. The method of claim 4, wherein said continuous data stream representative of said turbidity value corresponds to turbidity values associated with total iron content concentrations of from about 0 parts per billion (ppb) to about 20 ppb according to the linear relationship identified.

7. The method of claim 4, wherein said continuous data stream representative of said turbidity value corresponds to turbidity values associated with total iron content concentrations of from about 0 parts per billion (ppb) to about 50 ppb according to the linear relationship identified.

8. The method of claim 1, wherein said providing a high-intensity turbidimeter positioned in a water conduit of said high purity water system which is responsive to said turbidity of the water system comprises:
providing a laser turbidimeter at a water conduit having water exiting a filtration mechanism in a high pressure steam generated power plant.

9. The method of claim 8, wherein the water conduit having water exiting a filtration mechanism in a high pressure steam generated power plant comprises a water conduit leading to a steam formation mechanism in the high pressure steam generated power plant.

10. The method of claim 1, wherein said providing a high-intensity turbidimeter positioned in a water conduit of said high purity water system which is responsive to said turbidity of the water system comprises:
providing a laser turbidimeter at a water conduit having water exiting an ion exchange filtration purification mechanism in a high pressure steam generated power plant.

11. The method of claim 1, wherein said providing a high-intensity turbidimeter positioned in a water conduit of said high purity water system which is responsive to said turbidity of the water system comprises:
providing a laser turbidimeter at a water conduit having water exiting a condensing mechanism that condenses water from steam.

12. The method according to claim 1, further comprising:
providing a notification responsive to a determination that the calculated total iron content of the water exceeds a predetermined threshold value.

13. The method according to claim 12, further comprising:
diverting water of said conduit to an alternative path responsive to a determination that the calculated total iron content of the water exceeds a predetermined threshold value.
